(19) United States
(12) Patent Application Publication
(10) Pub. No.: US 2025/0060359 A1
(43) Pub. Date: Feb. 20, 2025

(54) FUSION PROTEINS AND METHODS FOR MONITORING FIBRONECTIN MATRIX ASSEMBLY

(71) Applicant: The Trustees of Princeton University, Princeton, NJ (US)

(72) Inventors: Jean E. Schwarzbauer, Princeton, NJ (US); Charles G. Miller, New York, NY (US); Henry A. Resnikoff, Princeton, NJ (US)

(73) Assignee: The Trustees of Princeton University, Princeton, NJ (US)

(21) Appl. No.: 18/804,806

(22) Filed: Aug. 14, 2024

Related U.S. Application Data
(60) Provisional application No. 63/532,589, filed on Aug. 14, 2023.

(51) Int. Cl. G01N 33/50; C07K 7/08; C12N 9/10

(52) U.S. Cl. CPC G01N 33/5032; C07K 7/08; C12N 9/1088; C12Y 205/01018; G01N 33/5044; C07K 2319/01; C07K 2319/60; G01N 2333/78

(57) ABSTRACT

Disclosed are methods and a pharmaceutical composition for the prevention and treatment of fibrosis by the inhibition of fibronectin matrix assembly, as well as insights into the molecular mechanism by which cell-mediated assembly of fibronectin (FN) matrix contributes to extracellular matrix (ECM) accumulation. Peptides S2, S3, S20, and variants thereof may be utilized, either alone or fused to one or more proteins. In one aspect, a construct comprising a first protein fused to a first end of a peptide and a second protein fused to a second end of the peptide may be used, where the peptide has at least 80% sequence identity to S2, S3, or S20. The peptide may then be allowed to interact with fibronectin.

### CROSS-REFERENCE TO RELATED APPLICATIONS

The present application claims priority to U.S. Provisional Patent Application No. 63/532,589 filed Aug. 14, 2023, the contents of which are incorporated by reference herein in its entirety.

### STATEMENT REGARDING FEDERALLY SPONSORED RESEARCH OR DEVELOPMENT

This invention was made with government support under Grant Nos. R01AR073236 and R01CA160611 awarded by the National Institutes of Health. The government has certain rights in the invention.

### STATEMENT REGARDING SEQUENCE LISTING

The instant application contains a Sequence Listing which has been submitted electronically in ST.26 XML file format, created on Jul. 12, 2024, is named PRIN84904_SL.xml and is 22,815 bytes in size. The ST.26 XML file is hereby incorporated by reference in its entirety.

### TECHNICAL FIELD

The present disclosure is drawn to techniques for preventing and treating fibrosis, and particularly, via inhibition of fibronectin matrix assembly.

### BACKGROUND

Fibrotic diseases are defined by the accumulation of extracellular matrix (ECM), which obstructs normal tissue function. In patients with diabetes, elevated glucose levels result in specific fibrotic changes in many organs, but the effects of diabetic fibrosis are particularly devastating in the eye and the kidney. Tight glucose control has been shown to reduce the progression of such complications, but there is currently no treatment that directly interferes with this fibrotic disease process.

At least one reason research has been slow in this area may be due to a lack of viable techniques for monitoring and/or diagnosing fibrosis matrix assembly.

### BRIEF SUMMARY

In various aspects, a fusion protein may be provided. The fusion protein may include a peptide having at least 80% sequence identity to GSYDWTKLPGLE (S2) [SEQ ID NO: 1]. The fusion protein may include a first protein fused to a first end (such as the N-terminal end) of the peptide. The fusion protein may include a second protein fused to a second end (such as the C-terminal end) of the peptide. The fusion protein may be less than about 100,000 Dalton molecular weight.

The first protein may be a purification tag. The purification tag may be, e.g., an epitope tag, a bacterial protein tag, a polyHistidine tag, or a protein with characterized binding partners for affinity purification. The purification tag may be Glutathione-S transferase (GST), Flag, cMyc, HA, or maltose binding protein (MBP). In a preferred embodiment, the purification tag is GST.

The second protein may be a fluorescent protein tag. The fluorescent protein tag may be, e.g., mPlum, mCherry, tdTomato, DsRed, mScarlet, mOrange, mKO, mCitrine, Venus, Ypet, EYFP, Emerald, green fluorescent protein (GFP) enhanced GFP (EGFP), CyPet, mCFPm, Cerulean, T-Sapphire, HaloTag, DsRed-Timer, or a combination thereof.

The fusion protein may include one or more additional moiety, such as one or more additional proteins, fused to the fluorescent tag (for example, fused to the fluorescent tag, away from the peptide).

In various embodiments, a composition may be provided. The composition may include a fusion protein as disclosed herein, and a carrier.

In various aspects, a method for monitoring and/or diagnosing fibrosis matrix assembly, or for artificially improving analysis of a fibronectin (FN) matrix, may be provided. The method may include providing a fusion protein as disclosed herein, and allowing the peptide to interact with fibronectin. The method may then include detecting the presence of the fusion protein at a location within or on the fibronectin.

### BRIEF DESCRIPTION OF FIGURES

The accompanying drawings, which are incorporated in and constitute a part of this specification, illustrate embodiments of the present invention and, together with a general description of the invention given above, and the detailed description of the embodiments given below, serve to explain the principles of the present invention.

FIG. 1 is a schematic illustration of a fusion protein.

FIG. 2 is an immunoblot comparing the effects of S3 and S20 against a control on the formation of FN matrix.

FIG. 3A is a graph showing the results from immunostaining different dosages of 2A1 monoclonal antibodies on FN matrix.

FIG. 3B is an immunoblot indicating the effect of different dosages of monoclonal antibody 2A1 on FN matrix.

FIGS. 4A-4C are representative photomicrographs of fixed and stained assembled matrices after clone 2 (1×10¹² pfu/mL) was incubated with NIH 3T3 fibroblasts for 2 days, and specifically stained for FN (4A) and phage (4B), as well as a merge of the two images (4C). Insets in 4A and 4B are maximum intensity projections of a single fibril obtained through confocal microscopy from the S2 condition for FN (4A) and phage (4B).

FIGS. 5A and 5B are graphical representations of two motifs identified in one example.

FIG. 6 is a series of images, where NIH 3T3 fibroblasts were seeded with 2.5 μg/mL S2 fusion protein and grown for 4 days on glass coverslips, where the images were collected every 24hours in the same location. The GFP fluorescence accumulates in a fibrillar pattern over time, revealing the accumulation of FN matrix.

FIGS. 7A-7D are images showing a GST-S2-GFP fusion protein identifying nascent fibrils, where NIH 3T3 cells were seeded on FN-coated glass coverslips and incubated for 8 hours with GST-S2-GFP, where the images include cells before (7A) and after (7B) the cells were fixed and stained for FN, and counterstained with DAPI (7C), then the GFP signal was visualized using innate fluorescence (7D), where co-localization reveals labeled nascent fibrils, as well as background due to the fusion protein interacting with the FN coat.

### DETAILED DESCRIPTION

The following description and drawings merely illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be only for illustrative purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or, unless otherwise indicated (e.g., “or else” or “or in the alternative”). Also, the various embodiments described herein are not necessarily mutually exclusive, as some embodiments can be combined with one or more other embodiments to form new embodiments.

The numerous innovative teachings of the present application will be described with particular reference to the presently preferred exemplary embodiments. However, it should be understood that this class of embodiments provides only a few examples of the many advantageous uses of the innovative teachings herein. In general, statements made in the specification of the present application do not necessarily limit any of the various claimed inventions. Moreover, some statements may apply to some inventive features but not to others. Those skilled in the art and informed by the teachings herein will realize that the invention is also applicable to various other technical areas or embodiments.

The ECM is a complex, highly ordered network of proteins that is assembled by cells and serves as a scaffold for cell attachment and organization; it is remodeled and maintained by cells and can itself influence cell behavior by binding to integrin receptors on the cell surface and by acting as a repository for growth factors. In many diseases, ECM accumulates erroneously, which obstructs normal tissue function and is broadly described as fibrosis.

There are numerous forms of fibrosis, including pulmonary fibrosis, skin fibrosis, cardiac fibrosis, kidney fibrosis, liver fibrosis, and more. While the diseases that trigger the various types of fibrosis to develop may vary (e.g., hepatitis may cause liver fibrosis, while lupus may trigger kidney fibrosis, etc.), all of the types of fibrosis include the same erroneous ECM accumulation concerns as disclosed above.

Chronically high blood sugar, a central feature of diabetes, is known to induce specific fibrotic changes, including excess ECM deposition surrounding capillaries. This fibrotic capillary thickening represents a widespread mechanism that underlies common diabetic complications including nephropathy, neuropathy, and diabetic retinopathy (DR).

As one example of these complications, DR is the most common cause of blindness in adults aged 20-74 and is expected to become even more prevalent as the rate of diabetes continues to rise. Currently, there are no therapies for preventing the onset or early progression of DR. The pathogenesis of DR is driven by high glucose induction of capillary dysfunction. The earliest detectable histological change in the diabetic retina is the thickening of the ECM that surrounds the retinal capillaries. Capillaries then become leaky, and loss of pericytes and endothelial cells occurs. Loss of normal capillary function results in a breakdown of the blood-retinal barrier (BRB), and the extravasation of fluid into the retinal tissue. Methods to prevent alterations of the BRB and ECM assembly are lacking. Understanding how ECM accumulation contributes to breakdown of the endothelial monolayer will be crucial to developing treatments for DR.

The retina is supplied by two networks of capillaries: one arising from the ophthalmic artery which forms multiple plexuses in the inner retina, and the other which supplies the outer retina from the choroid. The endothelial cells of the inner retinal capillary network are crucial to maintaining the BRB, which relies on the integrity of endothelial cell tight junctions mediating highly selective diffusion. Endothelial cells are supported by a BM composed of type IV collagen, laminin, nidogen, and perlecan. Endothelial cells assemble these ECM proteins on their basal side, but the proteins are secreted by multiple cell types in and around the capillary. The composition and organization of the BM is critical to the integrity of the endothelial monolayer and influences normal cell behavior by interacting with integrins that control cell signaling and cytoskeletal organization. However, the source of matrix proteins and the assembly of ECM in the retina is largely unexplored. In addition to the BM matrix proteins, endothelial cell behavior is affected by associated cell types (pericytes and astrocytes). Pericytes sit on the BM on the basal side of endothelial cells, and astrocytes are enriched in the retina in the vascularized zones and accompany the entire capillary structure. It is unclear how these cell types contribute to ECM assembly in the retina.

In DR, the BM loses its laminar organization and thickens significantly. FN, which is largely absent from the normal BM matrix, becomes highly abundant around the diabetic retina capillary. Blocking FN translation in the diabetic retina is sufficient to significantly reduce expansion of the BM matrix and to reduce loss of pericytes and endothelial cells, highlighting FN's role in diabetic dysregulation. The cellular sources of FN in the diabetic retina and the factors that impact its assembly in the retina are not well defined. Like cells in diabetic conditions in vivo, cells in culture also increase FN expression and assembly in high glucose. Recently a 3-cell co-culture system of retinal endothelial cells, pericytes and astrocytes has been developed to study endothelial barrier function as in the BRB. The researchers found that high glucose conditions increased the permeability of the endothelial cell monolayer in this model. The contribution of the subendothelial matrix to permeability was not determined but this culture system is amenable to analysis of ECM protein production and assembly. Alterations of BM matrix protein concentrations, including the overabundance of FN, are an underexplored potential driver of endothelial cell dysfunction in DR.

Formation of a BM requires coordinated assembly of multiple ECM proteins produced by several different cell types, but FN matrix assembly relies only on FN dimers binding to integrins. Force generated by the actin cytoskeleton pulls on the bound FN, changing its conformation and allowing for FN-FN interactions. These interactions lead to the formation of FN fibrils, which are converted into an insoluble network and become irreversible. FN is a foundational matrix protein, in that FN matrix is required for deposition of collagens and other ECM proteins. FN matrix also plays a critical role in development, wound healing, and cancer metastasis. In fibrotic states FN and collagen fibrils are abundant and disorganized. In DR, for example, while FN abundance in the BM increases dramatically, type IV collagen (Col IV), which is a normal component of the BM, is also significantly increased. In the mesangium, Col IV is co-assembled with FN in high glucose. FN-FN and FN-collagen interactions are critical for matrix assembly making the availability and abundance of FN and collagen-binding sites an active area of interest for understanding the dynamics of ECM accumulation by retinal cells and BM thickening in DR.

While DR represents an ocular disease with an early FN driven pathology, FN also plays a role in the later neovascularization of the retina, as FN matrix is crucial for blood vessel growth. Beyond DR, there are a number of other retinal pathologies that depend on FN matrix assembly such as proliferative vitreoretinopathy, age-related macular degeneration, and vitreomacular traction and macular holes (which have been treated by ocriplasmin cleavage of FN and laminin). Though FN matrix assembly drives numerous retinal pathologies, as well as fibrotic conditions throughout the body, there are currently no clinically relevant drugs that can directly inhibit the formation of ECM.

Thus, techniques for inhibiting the formation of ECM, or for aiding researchers to study the formation of such formations, are needed.

As disclosed herein, certain fusion proteins may be useful for various tasks related to monitoring and/or diagnosing fibronectin matrices.

Fusion Protein.

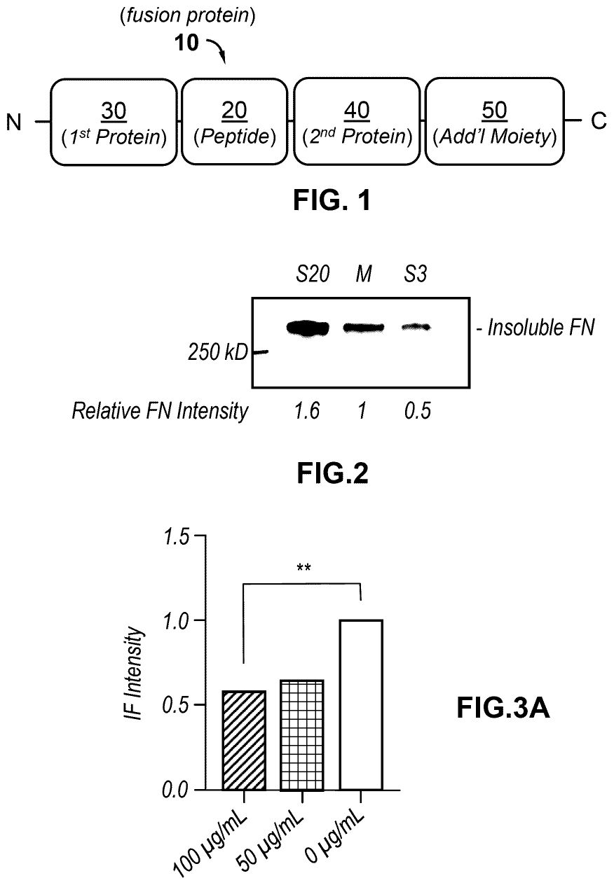

In various aspects, a fusion protein may be provided. Referring to FIG. 1, the fusion protein (10) may include a peptide (20) having at least 80% sequence identity to GSYDWTKLPGLE (S2) [SEQ ID NO: 1].

The fusion protein may include a first protein (30) fused to a first end (such as the N-terminal end) of the peptide. The first protein may be a purification tag. The purification tag may be, e.g., an epitope tag, a bacterial protein tag, a polyHistidine tag, or a protein with characterized binding partners for affinity purification. The purification tag may be Glutathione-S transferase (GST), Flag, cMyc, HA, or maltose binding protein (MBP). In a preferred embodiment, the purification tag is GST.

The fusion protein may include a second protein (40) fused to a second end (such as the C-terminal end) of the peptide. The second protein may include a fluorescent protein tag. The fluorescent protein tag may be, e.g., mPlum, mCherry, tdTomato, DsRed, mScarlet, mOrange, mKO, mCitrine, Venus, Ypet, EYFP, Emerald, green fluorescent protein (GFP) enhanced GFP (EGFP), CyPet, mCFPm, Cerulean, T-Sapphire, HaloTag, DsRed-Timer, or a combination thereof.

The fusion protein should have a molecular weight below a target threshold. That target threshold may be, e.g., no more than about 100,000 Dalton molecular weight. “About” as used with regards to molecular weight refers to weights that are +10% of the recited number. For example, a molecule that was “about 100 Dalton molecular weight” would include molecules from 90-110 Dalton molecular weight. In various embodiment, the fusion protein may be about 30 kDa - about 100 kDa, about 40 kDa-about 90 kDa, or about 50 kDa-about 80 kDa. In one preferred embodiment, the fusion protein may be no more than 90 kDa, no more than 80 kDa, no more than 70 kDa, or no more than 60 kDa.

The fusion protein may include one or more additional moicties (50), provided the overall molecular weight is maintained below the threshold as disclosed herein. The additional moieties may include, e.g., one or more additional proteins, fused to the fluorescent tag (for example, fused to the fluorescent tag, away from the peptide).

The additional protein(s) may include, e.g., one or more pharmaceutically active proteins (c.g., nucleic acid-based compounds intended to target a fibronectin matrix, etc.).

The additional protein(s) may include, e.g., a radiolabel. The term “radiolabel” refers to components where at least one of the atoms is a radioactive atom or a radioactive isotope, wherein the radioactive atom or isotope spontaneously emits gamma rays or energetic particles, for example alpha particles or beta particles, or positrons. Examples of such radioactive atoms include, but are not limited to, ³H (tritium), ¹⁴C, ¹¹C, ¹⁵O, ¹⁸F, ³²P, ³⁵S, ¹²³I , and ¹²⁵I. Any appropriate radiolabels known in the art may be utilized.

The additional proteins(s) may include one or more additional peptides. For example, referring to FIG. 2, it can be seen that S3 peptide inhibits matrix formation, and that S20 peptide enhances matrix formation.

Thus, the additional protein(s) may include, or may be free of, e.g., an inhibitor of a fibronectin (FN) self-association domain. The inhibitor may include NHVALWGTGTAS (S3) [SEQ ID NO: 2]. In some embodiments, the inhibitor may include a peptide having at least 90% sequence identity to S3. In some embodiments, the inhibitor may be a peptide having a single amino acid substitution mutation relative to S3. In some embodiments, the inhibitor may be a peptide having at least 80% sequence identity to S3. In some embodiments, the inhibitor may be a peptide having two amino acid substitution mutations relative to S3. In some embodiments, the inhibitor may have 11-13 amino acids. In some embodiments, the inhibitor may have 12 amino acids.

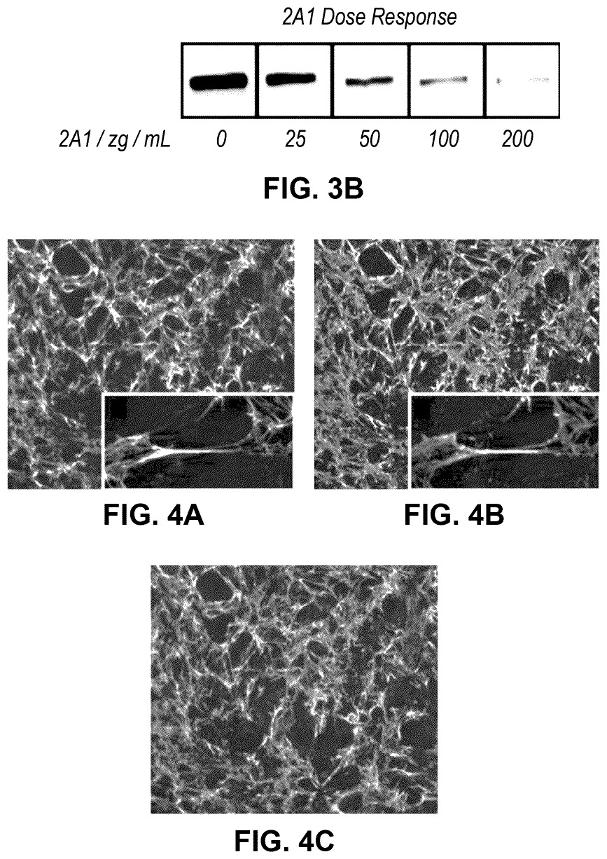

The inhibitor may be a monoclonal antibody, such as monoclonal antibody 1A2, 2A1, 3C4,3D5, 4C8, ctc. See, e.g., FIGS. 3A-3B.

Similarly, the additional protein(s) may include, or may be free of, e.g., a matrix formation enhancer. The matrix formation enhancer may include SDITPWWLLAQD (S20) [SEQ ID NO: 3]. In some embodiments, the matrix formation enhancer may include a peptide having at least 90% sequence identity to S20. In some embodiments, the matrix formation enhancer may be a peptide having a single amino acid substitution mutation relative to S20. In some embodiments, the matrix formation enhancer may be a peptide having at least 80% sequence identity to S20. In some embodiments, the matrix formation enhancer may be a peptide having two amino acid substitution mutations relative to S20. In some embodiments, the matrix formation enhancer may have 11-13 amino acids. In some embodiments, the matrix formation enhancer may have 12 amino acids.

Referring to FIG. 2, when incubated with cells during matrix assembly, S20 can enhance matrix formation by 60%.

Systems.

In some embodiments, the fusion proteins may be operably coupled (e.g., conjugated) to one or more other molecules, such as non-nucleic acid-based molecules. For example, in some embodiments, an S2-based fusion protein may be covalently bound to a photoabsorbing dye useful for near infrared photoimmunotherapy (NIR-PIT). Such dyes are well-known in the art, and may include, e.g., IR700 dye. With such approaches, the S2-based fusion protein would bind to the fibrin in, e.g., a cell microenvironment, such as a tumor cell microenvironment, and the molecule attached to the fusion protein would then interact as designed with that microenvironment. Compositions.

In various embodiments, a composition may be provided. The composition may include a fusion protein as disclosed herein, and a carrier.

The carrier may be, e.g., a pharmaceutically acceptable carrier. The term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

These compositions may also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly (orthoesters) and poly (anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Any such carrier, known to those of skill in the art, may be utilized. Considerations for the inclusion of various components in pharmaceutical compositions are described, e.g., in Brunton et al., “Goodman and Gilman's: The Pharmacological Basis of Therapeutics”, 13th Ed., McGraw Hill (2017), which is incorporated herein by reference in its entirety.

In some embodiments, the concentration of the fusion protein in the composition may be at least 0.1 μg/mL, at least 0.5 μg/mL, at least 1 μg/mL, at least 1.5 μg/mL, at least 2 μg/mL, at least 2.5 μg/mL, at least 5 μg/mL, or at least 10 μg/mL, up to no more than 500 mg/mL, no more than 250 mg/mL, no more than 100 mg/mL, no more than 1 mg/mL, or no more than 500 μg/mL, including all ranges and subranges thereof. In some embodiments, the concentration of the fusion protein in the composition may be no more than 20 μg/mL, no more than 10 μg/mL, or no more than 5 μg/mL. In some embodiments, the concentration of the fusion protein in the composition may be at least 100 μg/mL, or at least 200 μg/mL.

Method.

In various aspects, a method for monitoring and/or diagnosing fibrosis matrix assembly, or for artificially improving analysis of a fibronectin (FN) matrix, may be provided.

The method may include providing a fusion protein as disclosed herein (see FIG. 1).

The method may include allowing the fusion protein to interact with a fibronectin matrix. This may include allowing the sequence to interact with the matrix for a predetermined period of time. The predetermined period of time may be at least 1 hour. The predetermined period of time may be at least 4 hours. The predetermined period of time may be at least 8 hours. The predetermined period of time may be at least 12 hours. The predetermined period of time may be at least 24 hours. The predetermined period of time may be less than 72 hours. The predetermined period of time may be less than 48 hours.

Phage S2 and S2-based phages, effectively label fibrils as they are being assembled. As seen in FIGS. 4A-4C, incubation of fluorescent phage S2 with cells for 24 hours results in complete labeling of FN fibrils with incorporated phage. FIG. 4A shows the locations of the FN fibrils, the inset showing a confocal microscopy (100x, maximum intensity projection) of a single fibril. FIG. 4B shows the location of fluorescent phage S2, the inset showing a confocal microscopy (100x, maximum intensity projection) revealing colocalization of FN and phage, and FIG. 4C shows a merge of the two images, revealing highly colocalized staining of fibrils with an anti-FN antibody.

The method may include detecting the phage protein at a target location, where the presence at the location indicates the existence of the FN at the location and/or the relative amount of FN fibrils at a location. Thus, S2 and S2-based peptides can be used to identifying regions of increased matrix assembly.

S2, S3, and/or S20 can be identified using a M13 phage display 12-mer peptide library. The library can be screened by panning on surfaces coated with recombinant human 70 kD protein from stably transfected HEK 293 cells. After incubation, bound phages can be eluted using pH change, then amplified, titered, and panned additional times.

FN has been intensely studied, in both in vivo and in vitro models, to determine how FN matrix forms, what impact it has on various cells, and what role it plays in pathology. For all this intense study, researchers still lack tools to easily visualize FN matrix formation. FN matrix forms through the polymerization of multiple FN dimers into a fibrillar structure that can become entangled to produce a cohesive, 3D matrix. Removal of cells from this matrix does not destroy the structure, demonstrating the structural integrity of FN ECM. Antibodies, fluorescent conjugates, and genetically tagged FN have all been used to visualize FN matrix but have significant drawbacks. Antibody staining is typically conducted on fixed cells, meaning it is impossible to use live imaging to track a continuous process, such as matrix assembly. Genetically tagging ECM proteins is challenging, and in the case where it is accomplished, significant phenotypes can occur. Genetic tags also limit study of FN to the organisms/cells in which the tag exists, preventing more wide-reaching investigation. In contrast, fluorescently conjugated FN can be deployed in many systems, but cannot mark the native FN within the system, leading to incomplete labeling that can bias analysis. As such, a probe that could be deployed in diverse systems and allow for live imaging of FN would facilitate analysis of FN matrix and help further our understanding of FN in both biological and pathological contexts.

Pursuant to this goal, a phage display library was deployed to isolate peptides that bind FN. Phage display utilizes a diverse library of phages encoding unique protein sequences, in this case 12 amino acid (12aa) peptides. In the case of M13 phage display, the peptides are displayed as N-terminal fusions to the pIII protein, which exists in 5 copies at one end of M13, a filamentous phage. This leads to 5 copies of a single peptide displayed per phage. This is advantageous over higher valency systems such as PVIII fusions, which display thousands of copies of peptide per phage and often lead to recovery of lower affinity peptides. Through multiple rounds of panning and amplification, phage display leads to the identification of peptide sequences that bind the panned target protein. These peptides can then be synthetically fabricated, deployed as a phage-bound peptide, or fused to a larger construct.

The N-terminal 70kD fragment of FN (70kD) which contains the assembly domain and the collagen/gelatin binding domain was panned. This FN fragment was selected because of the intriguing possibility that ligand interaction with the assembly domain might influence FN matrix assembly, thus yielding a reagent that could modulate matrix accumulation. Furthermore, the gelatin binding domain acts as a native purification tag, allowing for one step affinity chromatography of recombinant 70kD. Furthermore, by panning a FN fragment instead of the full-length molecule, we have a better idea of where any resulting peptides might bind within FN.

Phage display for identifying FN binding peptides

Recombinant human 70kD protein was generated by stably transfecting a 70kD cDNA

expression vector into HEK293 cells. 70kD was isolated from conditioned media using gelatin-Sepharose affinity chromatography, which yielded >95% pure 70kD by Coomassie gel analysis.

Purified 70kD coated on plastic was used for panning a 12aa phage display library consisting of 109 unique 12aa peptides. The first round of library panning utilized 1011 phages, a 100-fold representation of the library diversity. Following washes utilizing detergent to remove low affinity or unbound phages, low pH was used to disrupt all interactions and recover the bound phages. These phages were amplified, and the amplified pool was panned two more times to enrich for 70kD binding.

Following 3 pans, the third eluted pool of phages was sequenced before the amplification step to reduce infectivity bias. Sequencing 50 phages from this pool yielded 26 unique peptide sequences with varying abundance, including sequences S2, S3, and S20. See Table 1, below.

TABLE 1

| Sequence | Clone Number |
|---|---|
| VSGYNVMPLNGD [SEQ ID NO: 4] | 1 |
| GSYDWTKLPGLE [SEQ ID NO: 1] | 2, 16, 31, 48 |
| NHVALWGTGTAS [SEQ ID NO: 2] | 3, 5, 27, 28 |
| HNQTMWWTFPST [SEQ ID NO: 5] | 4, 44 |
| SITEFNLLPGLW [SEQ ID NO: 6] | 6, 35 |
| AHBRLSGSAPMF [SEQ ID NO: 7] | 7, 22,38 |
| HITNWLSLSSYE [SEQ ID NO: 8] | 8 |
| HWSGTHALSGRA [SEQ ID NO: 9] | 9, 42 |
| GPMNNMYHGHFT [SEQ ID NO: 10] | 10 |
| DHVSLSGRSAAM [SEQ ID NO: 11] | 11 |
| DYSYFDLWYSDT [SEQ ID NO: 12] | 12, 29 |
| DHIQLSGASYFN [SEQ ID NO: 13] | 13, 30, 40 |
| HHIALSGSNSFG [SEQ ID NO: 14] | 14 |
| HQTYWMNLPGSY [SEQ ID NO: 15] | 15, 18 |
| DHSALRGTWTNR [SEQ ID NO: 16] | 17 |
| DHIPLRGQRVWQ [SEQ ID NO: 17] | 19, 24, 32, 41, 45 |
| SDITPWWLLAQD [SEQ ID NO: 3] | 20, 21, 46 |
| YVTNWGSLSGSY [SEQ ID NO: 18] | 23 |
| LETDWRSLSHMS [SEQ ID NO: 19] | 26 |
| IAYPTDMSLSYK [SEQ ID NO: 20] | 33 |
| DHIDVMGFTVFK [SEQ ID NO: 21] | 34 |
| HPTDWMNLPALS [SEQ ID NO: 22] | 36 |
| WHVALTGNNLER [SEQ ID NO: 23] | 37 |
| YSTTMWQYLPGA [SEQ ID NO: 24] | 39 |
| NHVPLSGFSRTP [SEQ ID NO: 25] | 47 |
| EVTNF [SEQ ID NO: 26] | 45 |

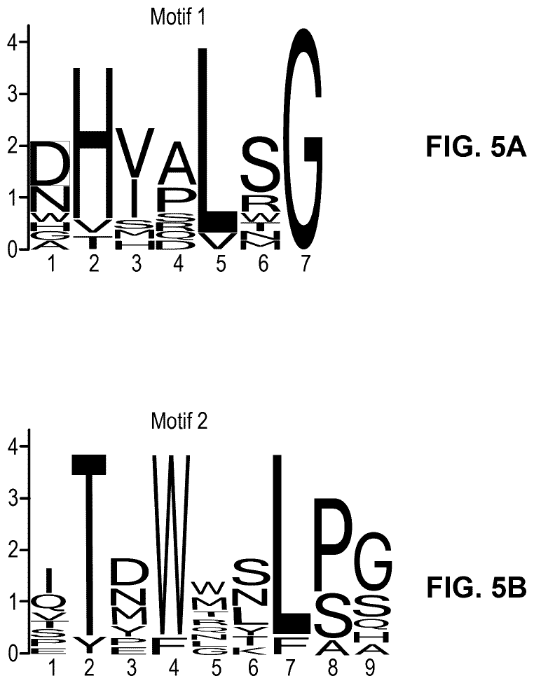

To search for potential sequence homology, the MEME suite motif-based sequence analysis tool was used. The MEME suite searches for un-gapped motifs, considering both amino acid identity and homology, and outputs an ordered list of motifs as well as a measure of statistical significance for each motif. 2 motifs were identified in the pool of 25 sequences. Motif 1 (see FIG. 5A), which spans 7 amino acids, was present in 12 sequences, and motif 2 (see FIG. 5B), which contains 9 amino acids, was present in 10 sequences. No isolated peptides contained both motifs, and 4 sequences did not contain either motif.

Single-letter abbreviations, along with size, are used to represent each amino acid in the motifs shown in FIGS. 5A and 5B in terms of frequency. For example, in motif 2, there is a tryptophan at position 4 in all but one of the 10 sequences that contain the motif. It is possible that the two motifs represent two distinct binding strategies for 2 locations on 70kD.

Evaluating peptide-FN interactions

Phage binding to 70kD was confirmed with an enzyme linked immunosorbent assay (ELISA) using 70kD-coated plastic as was used for the original phage panning experiment. We used a pool of unbound phage from the first pan as a negative control. All but 2 of the 26 unique clones were found to generate a positive ELISA signal, as defined by any mean signal higher than 3x the negative control signal. Both clones that did not register a positive ELISA signal are clones that did not fit into either motif.

Confluent NIH 3T3 fibroblasts, which have been well characterized to assemble a dense, fibrillar FN matrix, were used to test phage binding to FN fibrils. Cells were fixed, blocked, and incubated with individual phage clones, then co-stained with antibodies to M13 phage and FN and imaged. Most phage-monolayers looked like the negative control and contained infrequent phage signals that were not aligned with the FN matrix. Several of the clones generated a pattern of phage staining that had some co-localization with FN fibrils but also appeared to contain some phage aggregates. One clone, clone s2, created a fibrillar pattern that colocalized with the FN matrix better than the other phages.

To further analyze the interaction between the isolated phages and FN fibrils, each phage clone was incubated with growing fibroblast cultures that were actively assembling matrix. Monolayers were then stained for both phage and FN and resulting images were analyzed to assess the amount of overlap between phage and FN signal. As with the matrix binding experiment, the negative control phage showed very little association with FN fibrils. Comparison of equivalently adjusted merged images co-stained for phage and FN suggested some differences between phages in co-localization with FN. To quantify the extent of phage overlap with FN fibrils, Manders' coefficients were generated, which define the percent overlap between the two image channels. Manders' coefficients reveled the percent overlap of the phage fluorescence with FN was highest for clone S2 (Manders' coefficient 0.91). A summary of all Manders' coefficients and phage characterizations can be found in Table 2.

TABLE 2

| | ELISA | Matrix | Manders' | |
|---|---|---|---|---|
| Clone Number | signal | Binding | Coefficient | Motif |
| 1 | + | − | 0.253 | 1 |
| 2, 16, 31, 48 | + | + | 0.913 | 2 |
| 3, 5, 27, 28 | + | − | 0.527 | 1 |
| 4, 44 | + | − | 0.470 | 2 |
| 6, 35 | + | − | 0.606 | 2 |
| 7, 22, 38 | + | − | 0.390 | 1 |
| 8 | + | − | N/A | 2 |
| 9, 42 | + | − | 0.588 | 1 |
| 10 | − | − | N/A | |
| 11 | + | − | 0.173 | 1 |
| 12, 29 | + | + | 0.746 | |
| 13, 30, 40 | + | − | N/A | 1 |
| 14 | + | − | N/A | 1 |
| 15, 18 | + | − | N/A | 2 |
| 17 | + | − | N/A | 1 |
| 19, 24, 32, 41, 45 | + | − | 0.397 | 1 |
| 20, 21, 46 | + | − | 0.552 | 2 |
| 23 | + | − | 0.675 | 2 |
| 26 | + | − | N/A | 2 |
| 33 | − | − | 0.560 | |
| 34 | + | − | N/A | 1 |
| 36 | + | − | N/A | 2 |
| 37 | + | − | 0.698 | 1 |
| 39 | + | + | 0.677 | 2 |
| 47 | + | − | N/A | 1 |
| 45 | + | − | N/A | |

Given that the FN matrix did not appear saturated with bound phage in some samples, high concentration stocks were generated of two clones to see if incubating the monolayers with more phage would impact the amount of signal overlap. Clone 2 staining was extremely robust and images of matrix assembled in the presence of a higher concentration of phage appeared to have more coverage of fibrils by phage. In support of increased coverage was maintenance of the high Manders' coefficient. Clone 12, which had the second highest Manders' coefficient (see Table 2), appeared to have more phage-FN co-localization but the Manders' coefficient did not indicate higher coverage with inclusion of more phage, and matched the coefficient derived from earlier experiments. For clone 12, it appears that adding more phage led to higher background staining. Together the Manders' coefficients and merged images suggest that clone S2 gives more highly localized fibril staining. This is further supported by confocal images depicting a single fibril from a clone 2 incubation in which there is significant overlap between phage and FN at the level of a single fibril.

Beyond the ability of the isolated phage clones to bind FN matrix, their potential capacity to modulate matrix accumulation was considered. Given that the clones were panned against a FN fragment containing the assembly domain, it was questioned if they might modulate FN matrix assembly. To assess this, immunofluorescence was used to evaluate the FN matrix accumulation in the presence of phage clones that had demonstrated detectable matrix association. Interestingly, while no phages consistently repressed FN matrix accumulation, clone S20 increased FN matrix assembly by two-fold as evaluated by both immunofluorescence and immunoblots of DOC-insoluble FN, a matrix fractionation technique used to detect the amount of assembly.

Of further interest, it was found that clone S3 consistently decreased the insoluble fraction without repressing FN matrix immunofluorescence signal. While it was the best FN binding phage, clone S2 did not appear to impact FN matrix assembly by immunofluorescence or immunoblot analysis. These results demonstrate intriguing properties of select phage-displayed peptides, highlighting their potential as the foundation for FN-probing reagents.

Generating FN-binding fusion proteins

The 26 different peptides were initially characterized using amplified phage stocks. Most phage clones gave positive ELISA signals with the 70kD fragment but demonstrated undetectable or weak binding to FN matrix in cell monolayers. FN matrix assembly involves conformational change as well as applied tension to FN, so it is feasible that binding 70kD coated on plastic would not translate to binding assembled FN matrix. When incubated with fibroblasts as they assembled fibrils, several phages (e.g., 2, 12, 39) appeared to be incorporated into the fibrils resulting in a FN matrix uniformly labeled with phage. Two other phage clones (3 and 20) appeared to affect the level of matrix assembled. While preliminary experiments with phages suggested interesting peptide activities, interpretations were sometimes complicated by phage aggregation and stickiness giving high backgrounds and the inability to generate phage stocks at sufficiently high concentrations for dose-response analyses. Therefore, peptide sequences were transferred into a bacterial expression vector for further studies.

After defining three phages of interest (clones 2, 3, and 20), the 12aa peptide sequence from each phage (referred to as S2, S3, and S20, respectively) were cloned into an expression vector to generate fusion proteins to display the peptides. The peptides were fused to green fluorescent protein (GFP) to enable visualization and were cloned as N-terminal fusions to GFP to conserve the orientation with which they are displayed in the phage system (see FIG. 1). The fusion proteins also contain glutathione-S-transferase (GST) upstream of the peptide for purification, which can be cleaved using a tobacco etch virus (TEV) protease. The goal of this procedure was to generate stable reagents for probing and visualizing FN matrix assembly.

Following purification using glutathione affinity chromatography, the fusion proteins was tested in fibroblast culture to determine if they maintained the properties seen in the phage. It was found that the full-length fusion protein inclusive of the GST tag (GST-S2-GFP) maintained FN fibril binding activity. However, neither the S3 or S20 fusion proteins showed any matrix binding capacity or impact on matrix accumulation. Interestingly, cleavage of the GST tag from GST-S2-GFP completely ablated FN fibril binding, yielding no fluorescent signal. Addition of the cleaved GST tag along with the cleaved S2-GFP fusion in equimolar amounts did not rescue binding, and the GST-Control-GFP fusion alone, utilizing the control peptide sequence encoded by the pGEX4T3-GST-EGFP vector, does not bind FN fibrils. Furthermore, GST-S3-GFP and GST-S20-GFP showed no detectable FN fibril binding in culture, demonstrating that the FN fibril binding by the GST-S2-GFP construct is unique to the S2 peptide but requires the purification tag (here, GST). A concentration curve revealed 2.5 μg/mL to be one optimal concentration for labeling matrix in confluent fibroblast culture over a 24h growth period.

Evaluating FN-binding fusion proteins

It was investigated whether the GST-S2-GFP protein could be used to generate fluorescently tagged, decellularized ECM. To do this, NIH 3T3 cells were grown for four days with the GST-S2-GFP protein, until they assembled a robust, fluorescent matrix. The matrices were then decellularized using hypotonicity, high pH, and NP-40 detergent, and the resulting decellularized matrix were stained for FN. GFP fluorescence is maintained in a fibrillar pattern that aligns with the FN matrix. While the fibrillar structure of the GFP signal and the FN signal align, there is more variance in the GFP signal compared to the FN stain. Decellularized ECMs were then stored in PBS at 4° C. for eight days and re-examined. Matrices maintained innate, fibrillar GFP fluorescence, demonstrating that fusion protein remains bound to the fibrils and maintains its fluorescence through decellularization and storage.

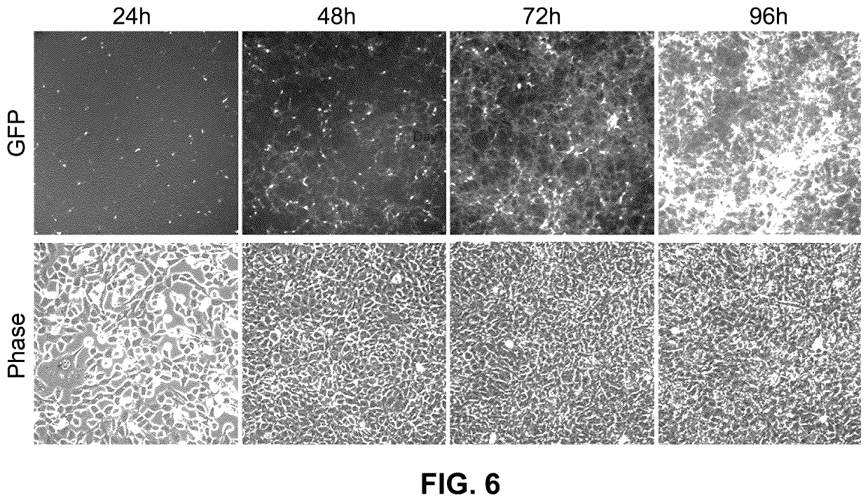

It was considered whether the S2-based fusion protein could also be used to track FN matrix accumulation over time in live cells. To test this, NIH 3T3 monolayers were grown for four days in the presence of GST-S2-GFP and imaged the same region every day (see FIG. 6). At 24hours, the subconfluent culture contained only a few bright foci. As time went on, matrix accumulated, ending with a dense ECM on day 4. Interestingly, the GST-S2-GFP signal did not accumulate uniformly and regions that had detectable signal at 48 and 72 hours had the most matrix at 96 hours, even though the monolayer by phase appears homogenous.

Because ECM is dynamic and FN fibril networks accumulate and are modified over time, it was considered whether the fusion protein could be used to detect initial FN fibrils even after the ECM continued to be synthesized and assembled. To test this, fibroblasts were grown for 48 hours post confluence, split into two-24 hr incubation periods with a scratch wound introduced at 24 hr. Exogenous human FN and GST-S2-GFP protein were include in the culture during the first 24 hr. During the second incubation period, one or both of these additions was omitted. During the second period, the scratch was filled in by cells that assembled a new matrix and this matrix would only be exposed to the factors supplied during the second incubation period. The monolayer was stained for human FN and DAPI following the second incubation period. For example, some cells were exposed to human FN and GST-S2-GFP for both the first and second growth period, and images detected no scratch but a continuous matrix labeled with S2 fusion protein and anti-human FN antibodies. For other cells, after the scratch, the cells were exposed to only human FN during the second growth period, leading to a continuous human FN matrix including within the scratched area, but GST-S2-GFP fibrils were only detected in the unscratched area. In still other cells, neither protein was included in the second incubation period and colocalized GFP and human FN fibrils were detected in the unscratched area. The matrix filling in the scratch was not detected without added GST-S2-GFP or human FN. These experiments demonstrate that the fibrils that become associated with GST-S2-GFP remain fluorescent during the 24-hour period where the fusion protein is removed, allowing us to label fibrils from the first growth period and visualize them within the context of the matrix that has continued to evolve during the second growth period.

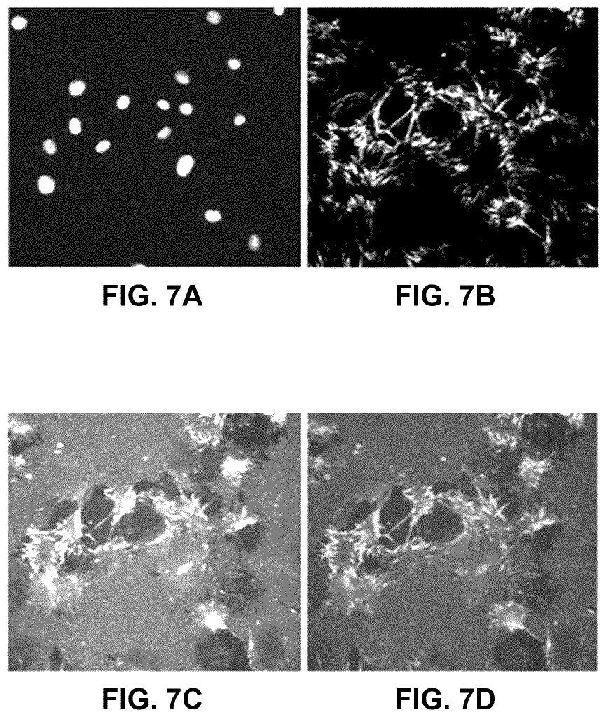

Having examined a dense FN matrix in multiple contexts, nascent FN fibrils were then probed to investigate if GST-S2-GFP can identify the earliest FN fibrils assembled in addition to dense matrix. Cells were seeded sparsely on bovine FN-coated glass coverslips and incubated with GST-S2-GFP and human FN for 8 hours. Nascent fibrils were detected by staining for human FN, which does not stain the FN coat. GFP fluorescence highlights and co-localizes with nascent fibrils and is detectable even above the significant background from the fusion protein binding to the FN coat (sec FIGS. 7A-7D). The GFP moiety of the fusion protein is not essential for fibril binding. A GST-S2-mScarlet protein and found that it also co-localizes with FN fibrils assembled by fibroblasts similarly to the GFP protein. Similar results were found when probing a HUVEC monolayer FN.

Given that FN matrix assembly occurs in many pathological states, including cancer and fibrosis, it would be useful to easily detect sites of FN assembly or track the accumulation of FN matrix in real time. As such, developing tools to probe FN matrix will be useful for visualizing and analyzing matrix accumulation in cell-based models of disease, and the contribution of FN matrix to other cellular changes. Using phage display, peptides were isolated that bind the N-terminal 70kD fragment of FN, and used one of these peptides, S2, to generate a novel, GFP-tagged FN-binding protein for visualizing FN matrix. The fusion protein, such as the GST-S2-GFP fusion protein used in this example, can identify FN matrix accumulation and nascent FN fibrils and interacts with fibrils to form a stable complex that resists dissociation during decellularization. The S2 peptide-based fusion protein can also be utilized for live imaging of FN fibrils and identification of fibril assembly in an existing FN matrix. The fusion protein is a new tool for studying FN matrix and could be repurposed or reconfigured to target FN matrix in vivo for research or therapeutic purposes.

S2 is required for matrix binding, but there may be additional elements that help localize teh protein to the matrix. For example, the presence of a moiety at the N-terminus of the peptide or the purification tag (and particularly the GST tag) may be aiding to localize the protein to the matrix. Further, it could be that the conformation of the peptide is influenced by the GST moiety.

The fusion protein can be used in live imaging to follow matrix accumulation. Repetitive imaging of the same location over time in fibroblast cultures revealed increases in fibril density, beginning with bright foci and faint fibrils and progressing to a dense sheet of FN fibrils. Beyond these applications, it is conceivable that these fusion proteins could be used to target FN in other assays. S2 fusion protein could be utilized in high throughput screens to detect matrix accumulation directly through fluorescence accumulation. Such assays could be used to identify inhibitors of FN matrix assembly, using fusion protein fluorescence accumulation as a readout. Furthermore, addition of fusion protein to cell culture could be used to live image tagged cell lines to study the relationship between FN matrix and tagged proteins of interest, such as focal adhesion associated proteins. A combination of GFP and mScarlet fusion proteins might be used in a pulse-chase assay to visualize the FN assembly that occurs before and after the addition of a stimulating agent or inhibitor.

Peptide-based targeting of drugs to FN matrix has been successfully employed to enhance therapy, and several groups have generated FN binding peptides to address research questions pertaining to FN matrix. S2 peptide-based proteins have a high specificity for FN and stay well associated with FN fibrils once bound, characteristics that support favorable pharmacodynamics. Thus, as will be understood in the art, a drug bound to S2-based fusion protein could accumulate at sites of FN matrix assembly, allowing for delivery of therapeutic compounds in, e.g., cancer, fibrosis, or other FN rich disease states. The retina could be an ideal tissue for targeting FN matrix, given the relative ease of drugging the retina compared to other organs. A radiolabel could also be used to identify sites of FN matrix accumulation via PET scans, potentially allowing for the identification of early disease states such as metastatic niches. While significant testing and optimization would be needed to achieve these goals, our research represents an important first step toward generating reagents to target FN in a diagnostic or therapeutic capacity.

Various modifications may be made to the systems, methods, apparatus, mechanisms, techniques, and portions thereof described herein with respect to the various figures, such modifications being contemplated as being within the scope of the invention. For example, while a specific order of steps or arrangement of functional elements is presented in the various embodiments described herein, various other orders/arrangements of steps or functional elements may be utilized within the context of the various embodiments. Further, while modifications to embodiments may be discussed individually, various embodiments may use multiple modifications contemporaneously or in sequence, compound modifications and the like.

Although various embodiments which incorporate the teachings of the present invention have been shown and described in detail herein, those skilled in the art can readily devise many other varied embodiments that still incorporate these teachings. Thus, while the foregoing is directed to various embodiments of the present invention, other and further embodiments of the invention may be devised without departing from the basic scope thereof. As such, the appropriate scope of the invention is to be determined according to the claims.

What is claimed is:

1. A fusion protein, comprising:
a peptide having at least 80% sequence identity to GSYDWTKLPGLE (S2) [SEQ ID NO: 1];
a first protein fused to a first end of the peptide; and
a second protein fused to a second end of the peptide;
wherein the fusion protein is less than about 100,000 Dalton molecular weight.

2. The fusion protein of claim 1, wherein the first protein comprises a purification tag.

3. The fusion protein of claim 2, wherein the purification tag is an epitope tag, a bacterial protein tag, a polyHistidine tag, or a protein with characterized binding partners for affinity purification.

4. The fusion protein of claim 3, wherein the purification tag is Glutathione-S transferase (GST), Flag, cMyc, HA, or maltose binding protein (MBP).

5. The fusion protein of claim 2, wherein the first end is the N-terminal end of the peptide.

6. The fusion protein of claim 2, wherein the first protein is glutathione-S-transferase.

7. The fusion protein of claim 1, wherein the second protein comprises a fluorescent protein tag.

8. The fusion protein of claim 7, wherein the fluorescent protein tag is mPlum, mCherry, tdTomato, DsRed, mScarlet, mOrange, mKO, mCitrine, Venus, Ypet, EYFP, Emerald, green fluorescent protein (GFP) enhanced GFP (EGFP), CyPet, mCFPm, Cerulean, T-Sapphire, HaloTag, DsRed-Timer, or a combination thereof.

9. The fusion protein of claim 7, wherein the second end is the C-terminal end of the peptide.

10. The fusion protein of claim 7, further comprising at least a third protein fused to the fluorescent tag.

11. The fusion protein of claim 10, wherein the third protein is a pharmaceutically active protein.

12. A composition comprising:
The fusion protein of claim 1; and
a carrier.

13. A method for monitoring and/or diagnosing fibrosis matrix assembly, or artificially improving analysis of a fibronectin (FN) matrix, comprising:
providing a fusion protein comprising:
a peptide having at least 80% sequence identity to GSYDWTKLPGLE (S2) [SEQ ID NO: 1];
a first protein fused to a first end of the peptide; and
a second protein fused to a second end of the peptide;
wherein the fusion protein is less than about 100,000 Dalton molecular weight; and
allowing the peptide to interact with fibronectin.

14. The method of claim 13, further comprising detecting the presence of the fusion protein at a location within or on the fibronectin.


SEQUENCE LISTING (26 sequences)

SEQ ID NO: 1 - protein, 12 aa, unidentified
GSYDWTKLPGLE

SEQ ID NO: 2 - protein, 12 aa, unidentified
NHVALWGTGTAS

SEQ ID NO: 3 - protein, 12 aa, unidentified
SDITPWWLLAQD

SEQ ID NO: 4 - protein, 12 aa, unidentified
VSGYNVMPLNGD

SEQ ID NO: 5 - protein, 12 aa, unidentified
HNQTMWWTFPST

SEQ ID NO: 6 - protein, 12 aa, unidentified
SITEFNLLPGLW

SEQ ID NO: 7 - protein, 12 aa, unidentified
AHBRLSGSAPMF

SEQ ID NO: 8 - protein, 12 aa, unidentified
HITNWLSLSSYE

SEQ ID NO: 9 - protein, 12 aa, unidentified
HWSGTHALSGRA

SEQ ID NO: 10 - protein, 12 aa, unidentified
GPMNNMYHGHFT

SEQ ID NO: 11 - protein, 12 aa, unidentified
DHVSLSGRSAAM

SEQ ID NO: 12 - protein, 12 aa, unidentified
DYSYFDLWYSDT

SEQ ID NO: 13 - protein, 12 aa, unidentified
DHIQLSGASYFN

SEQ ID NO: 14 - protein, 12 aa, unidentified
HHIALSGSNSFG

SEQ ID NO: 15 - protein, 12 aa, unidentified
HQTYWMNLPGSY

SEQ ID NO: 16 - protein, 12 aa, unidentified
DHSALRGTWTNR

SEQ ID NO: 17 - protein, 12 aa, unidentified
DHIPLRGQRVWQ

SEQ ID NO: 18 - protein, 12 aa, unidentified
YVTNWGSLSGSY

SEQ ID NO: 19 - protein, 12 aa, unidentified
LETDWRSLSHMS

SEQ ID NO: 20 - protein, 12 aa, unidentified
IAYPTDMSLSYK

SEQ ID NO: 21 - protein, 12 aa, unidentified
DHIDVMGFTVFK

SEQ ID NO: 22 - protein, 12 aa, unidentified
HPTDWMNLPALS

SEQ ID NO: 23 - protein, 12 aa, unidentified
WHVALTGNNLER

SEQ ID NO: 24 - protein, 12 aa, unidentified
YSTTMWQYLPGA

SEQ ID NO: 25 - protein, 12 aa, unidentified
NHVPLSGFSRTP

SEQ ID NO: 26 - protein, 5 aa, unidentified
EVTNF